(19) United States
(12) Patent Application Publication
(10) Pub. No.: US 2013/0129827 A1
(43) Pub. Date: May 23, 2013

(54) EXTENDED RELEASE PHARMACEUTICAL COMPOSITIONS OF PALIPERIDONE AND PROCESSES OF PREPARATION THEREOF

(71) Applicants: Kumaravel Vivek, Chennai (IN); Rajan Kumar Verma, New Delhi (IN); Romi Barat Singh, Varanasi (IN)

(72) Inventors: Kumaravel Vivek, Chennai (IN); Rajan Kumar Verma, New Delhi (IN); Romi Barat Singh, Varanasi (IN)

(73) Assignee: RANBAXY LABORATORIES LIMITED, New Delhi, Delhi (IN)

(21) Appl. No.: 13/502,016

(22) PCT Filed: Oct. 18, 2010
(86) PCT No.: PCT/IB10/54714

§ 371 Date: Dec. 19, 2012

(30) Foreign Application Priority Data
Oct. 16, 2009 (IN) 2150/DEL/2009

(51) Int. Cl. A61K 9/00

(52) U.S. Cl. CPC A61K 9/0002
USPC 424/487; 424/484; 514/259.41; 424/488; 424/486; 424/400; 427/2.21

(57) ABSTRACT

The present invention relates to extended release pharmaceutical compositions of paliperidone and process of preparation thereof.

### FIELD OF THE INVENTION

The present invention relates to extended release pharmaceutical compositions of paliperidone and process of preparation thereof.

### BACKGROUND OF THE INVENTION

Paliperidone as disclosed in U.S. Pat. No. 5,158,952, is chemically (±)-3-[2-[4-(6-fluoro-1,2-benzisoxazol-3-yl)-1-piperidinyl]ethyl]-6,7,8, 9-tetrahydro-9-hydroxy-2-methyl-4H-pyrido[1,2-a]pyrimidin-4-one. Paliperidone is a psychotropic agent which belongs to the chemical class of benzisoxazole derivatives, and is an active metabolite of risperidone. It differs from risperidone and related prior art by its substitution on the 1-position of the piperidine moiety. Extended release osmotic tablets of paliperidone are commercially available in USA, in 1.5, 3, 6 and 9 mg strengths, under the trade name Invega®.

Paliperidone has a long half-life of about a day and therefore is not a typical candidate for the extended delivery. However, immediate release compositions cause side effects such as anxiety, somnolence, dizziness, constipation, and/or extrapyramidal symptoms due to high blood plasma concentration levels, thereby restricting its use. In order to obtain a therapeutic effect with reduced side effects, drug plasma concentrations need to be sustained above a minimum pharmacodynamic concentration and below the threshold maximum tolerable concentrations. In this regard, extended release pharmaceutical compositions of paliperidone are desirable over the immediate release compositions.

A review of the art shows that a PCT Publication No. WO 00/35419 teaches osmotic dosage forms of risperidone, further U.S. Application Publication No. 2005/0232995 teaches capsule shaped osmotic dosage forms of paliperidone, providing substantiality ascending release rate for a prolonged period of time.

For providing the extended release of a drug, apart from the osmotic dosage forms, diffusion systems such as reservoir and matrix diffusion systems, dissolution systems such as matrix dissolution systems and encapsulated dissolution systems, combination of diffusion/dissolution systems, complexation and ion-exchange resin systems are the other available alternative systems. These systems are easy to manufacture and are relatively simpler and cost effective over the osmotic dosage forms. Our co-pending Indian Application No. 424/DEL/2008 discloses diffusion controlled matrix system or dissolution controlled encapsulated system or combination of these to provide the desired in-vitro and in-vivo release profiles. Further, U.S. Application No. 2009/0087487 discloses the extended release dosage form of paliperidone, which includes at least a first component and a second component located adjacent to the first component, wherein the first component comprises at least one delay layer comprising a polymer, and the second component comprises non-coated paliperidone.

There still exists a need of alternative pharmaceutical compositions of paliperidone having desired in-vitro and in-vivo release profiles, which can be economically and industrially manufactured.

We have now formulated extended release pharmaceutical compositions of paliperidone and processes of preparation thereof, achieving the desired in-vitro and in-vivo release profiles.

### SUMMARY OF THE INVENTION

In one general aspect, the present invention provides for an extended release pharmaceutical composition that includes paliperidone and one or more release controlling polymers in a matrix formulation, coated with one or more delayed release coatings.

Embodiments of this aspect of the invention may include one or more of the following features. For example, the paliperidone is present in an amount of about 0.5% to about 10% w/w of the total composition. The paliperidone particles may have a D₅₀ of about 1 μm to about 10 μm and a D₉₀ of about 2 μm to about 30 μm.

The one or more release controlling polymer may include a water insoluble polymer. The one or more release controlling polymer may be present in an amount of about 10% to about 90% w/w of the total composition.

Suitable release controlling polymers include one or more of ethyl cellulose, hydroxyethylcellulose, cellulose acetate, cellulose acetate butyrate, cellulose acetate phthalate, cellulose acetate trimellitate, hydroxypropyl methylcellulose phthalate, poly (alkyl) methacrylate, and copolymers of acrylic or methacrylic acid esters, ammonio methyacrylate copolymer, methyacrylic acid copolymers, methacrylic acid-acrylic acid ethyl ester copolymer, methacrylic acid esters neutral copolymer, dimethylaminoethylmethacrylate-methacrylic acid esters copolymer, vinyl methyl ether/maleic anhydride copolymers, their salts and esters, polyvinyl acetate and mixtures thereof.

The extended release pharmaceutical composition may further include one or more pharmaceutically inert excipients selected from the group of solubility enhancers/solubilizers, fillers, binders, lubricant/glidants coloring agents, plasticizers and opacifiers. The extended release pharmaceutical composition may also include one or more non-functional coating layers.

The extended release pharmaceutical composition may release the paliperidone at a rate of:

(a) not more than 20% after 2 hours;

(b) between 10 and 40% after 8 hours;

(c) between 40 and 80% after 14 hours;

(d) between 70 and 95% after 18 hours; and

(e) more than 80% after 24 hours.

In another general aspect, the present invention provides for a process for the preparation of the extended release pharmaceutical composition, wherein said process includes the steps of:
(a) blending the paliperidone, the one or more release controlling polymers with one or more pharmaceutically inert excipients;
(b) optionally granulating the blend of step (a);
(c) compressing the blend/granules of steps (a) or (b) into tablets using appropriate tooling; and
(d) applying one or more delayed release coatings on to the compressed tablets of step (c).

In another general aspect, the present invention provides for a process for the preparation of the extended release pharmaceutical composition, wherein said process includes the steps of:
(a) granulating the paliperidone with the one or more release controlling polymers, with or without one or more pharmaceutically inert excipients;
(b) mixing the granules with the one or more release controlling polymers and one or more pharmaceutically inert excipients;
(c) compressing the granules into tablets using appropriate tooling; and
(d) applying the one or more delayed release coatings on to the compressed tablets of step (c).

In yet another general aspect, the present invention provides for a process for the preparation of the extended release pharmaceutical composition, wherein said process includes the steps of:
(a) granulating the paliperidone with the one or more release controlling polymers, with or without one or more pharmaceutically inert excipients;
(b) mixing the granules with the one or more release controlling polymers and one or more pharmaceutically inert excipients;
(c) compressing the granules into tablets using appropriate tooling;
(d) applying the one or more delayed release coatings on to the compressed tablets of step (c);
(e) mixing the paliperidone with one or more coating additives; and
(f) applying the mixture of step (e) on to the coated tablets of step (d).

In another general aspect, the present invention provides for a process for the preparation of the extended release pharmaceutical composition, wherein said process includes the steps of:
(a) blending the paliperidone and the one or more release controlling polymer with one or more pharmaceutically inert excipients;
(b) optionally granulating the blend of step (a);
(c) compressing the blend/granules of steps (a) or (b) into tablets using appropriate tooling; and
(d) applying the compression coating of the one or more delayed release coatings on to the compressed tablets of step (c).

In another general aspect, the present invention provides for a process for the preparation of the extended release pharmaceutical composition, wherein said process includes the steps of:
(a) blending the paliperidone with one or more pharmaceutically inert excipients;
(b) optionally granulating the blend of step (a);
(c) compressing the blend/granules of steps (a) or (b) into tablets using appropriate tooling; and
(d) mixing the paliperidone with the one or more release controlling polymers and one or more coating additives; and
(e) applying the compression coating of the mixture of step (d) on to the compressed tablets of step (c).

In another general aspect, the present invention provides for a process for the preparation of the extended release pharmaceutical composition, wherein said process includes the steps of:
(a) blending the paliperidone with one or more pharmaceutically inert excipients;
(b) optionally granulating the blend of step (a);
(c) compressing the blend/granules of steps (a) or (b) into tablets using appropriate tooling; and
(d) applying a compression coating of the one or more delayed release coatings on to the compressed tablets of step (c).

In another general aspect, the present invention provides for a method of treatment of neurological disorders in mammals, which includes administering to a mammal in need thereof, an extended release composition that includes paliperidone or pharmaceutically acceptable salts, enantiomers, hydrates, solvates, metabolites, prodrugs or mixture thereof in one or more release controlling polymers, coated with one or more delayed release coatings.

### DETAILED DESCRIPTION OF THE INVENTION

The term “paliperidone” as used herein includes paliperidone as well as pharmaceutically acceptable salts, enantiomers, hydrates, solvates, metabolites, prodrugs or mixture thereof. The amount of paliperidone may vary from about 0.5% to about 10% w/w of the total pharmaceutical composition. Paliperidone particles used in the present invention have a D₅₀ value in range of about 1 μm to about 10 μm and a D₉₀ in the range of about 2 μm to about 30 μm.

The extended release pharmaceutical tablet of paliperidone of the present invention may be bioequivalent to that of the commercially available Invega® tablets.

The extended release pharmaceutical composition of paliperidone of the present invention includes a matrix of paliperidone in one or more release controlling polymers. The controlled release polymer is a water insoluble polymer selected from the group including ethyl cellulose, hydroxyethylcellulose, cellulose acetate, cellulose acetate butyrate, cellulose acetate phthalate, cellulose acetate trimellitate, hydroxypropyl methylcellulose phthalate, poly (alkyl) methacrylate, and copolymers of acrylic or methacrylic acid esters, ammonio methyacrylate copolymer (Eudragit® RL or Eudragit® RS), methyacrylic acid copolymers (Eudragit® L or Eudragit® S), methacrylic acid-acrylic acid ethyl ester copolymer (Eudragit® L 100-5), methacrylic acid esters neutral copolymer (Eudragit® NE 30D or Eudragit® NM 30D), dimethylaminoethylmethacrylate-methacrylic acid esters copolymer (Eudragit® E 100), vinyl methyl ether/maleic anhydride copolymers, their salts and esters (Gantrez™), polyvinyl acetate and mixtures thereof.

The term “delayed release coating” includes one or more release controlling polymers selected from the group comprising cellulose derivatives such as ethyl cellulose, butyl cellulose, hydroxypropyl methyl cellulose, hydroxypropyl cellulose, ethyl hydroxyethyl cellulose, carboxymethyl cellulose, cellulose acetate butyrate, cellulose acetate phthalate, cellulose acetate, cellulose propionate; polysaccharides, like alginate; xanthan; carrageenan; scleroglucan; pullulan; dextran; haluronic acid; chitin; chitosan; starch; other natural polymers, like proteins (e.g. albumin, gelatine); natural rubber; synthetic polymers, like acrylates (e.g. polymethacrylate, poly(hydroxy ethyl methacrylate), poly(methyl methacrylate), poly(hydroxy ethyl methacrylate-co methyl methacrylate), methacrylate copolymers with trimethyl-aminoethyl-methacrylate (such as Eudragit® RL, Eudragit® RS and Eudragit® NE, Carbopol® 934), ethyl acrylate-methyl methacrylate-ethyl ammonium trimethyl chloride methacrylate copolymer and N-vinyl-2-pyrrolidone cellulose ether; cellulose ester; polyvinyl ester; acrylic acid type polymer having a quaternary ammonium-alkyl group; and Plasdone™ K-90, homopolymer of N-vinyl-2-pyrrolidone; polyvinylpyrolidone; ethyl acryalte-methyl methacrylate-ethyl ammonium trimethyl chloride methacrylate copolymer and N-vinyl-2-pyrrolidone cellulose ether, cellulose ester; polyamides (e.g. polyacrylamide, poly(methylene bisacrylamide)); polyanhydrides (e.g. poly(bis carboxyphenoxy)methane); PEO-PPO block-co-polymers (e.g. poloxamers, etc.); polyvinyl chloride; polyvinyl pyrrolidone; polyvinyl acetate; polyvinyl butyrate; polyvinyl alcohol; polyethylene, polyethylene glycols and co-polymers thereof; polyethylene oxides and co-polymers thereof; polypropylene and co-polymers thereof; polystyrene; polyesters (e.g. poly(lactic acid), poly(glycolic acid), poly(caprolactone), etc, and co-polymers thereof, and poly(ortho esters), and co-polymers thereof; resins (e.g. Dowen™, Amberlite®); polycarbonate; cellophane; silicones (e.g. poly(dimethylsiloxane)); polyurethanes; synthetic rubbers (e.g. styrene butadiene rubber, isopropene rubber); others, like shellacs; waxes (e.g. carnauba wax, beeswax, glycowax, castor wax); nylon; stearates (e.g. glycerol palmitostearate, glyceryl monostearate, glyceryl tristearate, stearyl alcohol); lipids (e.g. glycerides, phospholipids); paraffin; cetyl alcohol, in particular polyethylene oxide.

The term “polyethylene oxide” as used herein is a non-ionic homopolymer of the formula —(—O—CH₂—CH₂—)ₙ—, wherein n represents the average number of oxyethylene groups, n generally being from about 2,000 to about 100,000. It is a water soluble resin which is available as a white powder in several grades having different molecular weights which vary in viscosity profile when dissolved in water. Polyethylene oxide resin is commercially available under the trade name Polyox® from the Union Carbide Corporation. Polyox® WSR 303 has an average molecular weight of about 5,000,000 to 6,000,000, and a 1% aqueous solution thereof at 25° C. has a viscosity of 7,200 to 10,000 cps on a Brookfield RVF, No. 2 spindle at 2 rpm, and a pH of 8 to 10. It is contemplated that mixtures of varying molecular weight polymers may also be employed as a matrix system to obtain the desired tablet release properties.

The pharmaceutical tablet of paliperidone may further include one or more pharmaceutically inert excipients which may be selected from solubility enhancers/solubilizers, fillers, binders, lubricants/glidants coloring agents, plasticizers and opacifiers.

Suitable solubility enhancers include polyethylene glycols, surfactants, propylene glycol, glycerol, mono-alcohols, higher alcohols, DMSO, dimethylformamide, N. N-dimethylacetamide, 2-pyrolidone, N-(2-hydroxyethyl)pyrrolidone, N-methylpyrrolidone, 1-dodecylazacycloheptan-2-one and other n-substituted-alkyl azacycloalkyl-2-ones, preferably polyethylene glycol.

Suitable fillers or diluents include lactose, pregelatinized starch, calcium carbonate, calcium phosphate dibasic, calcium phosphate tribasic, calcium sulphate, kaolin, starch, and the like.

Suitable lubricants/glidants include colloidal silicon dioxide, stearic acid, magnesium stearate, calcium stearate, zinc stearate, sodium stearyl fumarate, talc, hydrogenated castor oil, sucrose esters of fatty acid, microcrystalline wax, yellow beeswax, white beeswax, and the like.

Suitable binders include methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, sodium alginate, propylene glycol, and the like.

Suitable coloring agents includes any FDA approved color for oral use.

Suitable plasticizers include triethylcitrate, dibutylsebacate, acetylated triacetin, tributylcitrate, glyceroltributyrate, monoglyceride, rape oil, olive oil, sesame oil, acetyltributylcitrate, acetyltriethylcitrate, glycerin sorbitol, diethyloxalate, diethyl phthalate, diethylmalate, diethylfumarate, dibutylsuccinate, diethylmalonate, dioctylphthalate and the like.

Suitable opacifiers include titanium dioxide, manganese dioxide, iron oxide, silicon dioxide, and mixtures thereof

In one of the embodiments, the pharmaceutical tablet of present invention may be prepared by the conventional techniques known in the art, such as, wet granulation, dry granulation, direct compression or extrusion-spheronization or hot melt extrusion. The wet granulation process involves the use of water or any other suitable granulating fluid. Dry granulation may involve use of roller compacter or any suitable technique.

Suitable granulating fluid/solvents for coating include acetone, ethanol, isopropyl alcohol, methylene chloride or combination thereof.

The pharmaceutical composition of the present invention may be further coated with one or more non-functional coating layers, if desired, including film forming polymers with/without coating additives.

Suitable coating additives include plasticizers, coloring agents, lubricants/glidants, and the like.

Suitable film-forming polymers include ethylcellulose, hydroxypropyl methylcellulose, hydroxypropylcellulose, methylcellulose, carboxymethyl cellulose, hydroxymethylcellulose, hydroxyethylcellulose, cellulose acetate, hydroxypropyl methylcellulose phthalate, cellulose acetate phthalate, cellulose acetate trimellitate; waxes such as polyethylene glycol; methacrylic acid polymers such as Eudragit®; and the like. Alternatively, commercially available coating compositions comprising film-forming polymers marketed under various trade names, such as Opadry®, may also be used.

Suitable plasticizers include triethylcitrate, acetylated triacetin, tributylcitrate, glyceroltributyrate, monoglyceride, rape oil, olive oil, sesame oil, acetyltributylcitrate, acetyltriethylcitrate, glycerin sorbitol, diethyloxalate, diethyl phthalate, diethylmalate, diethylfumarate, dibutylsuccinate, diethylmalonate, dioctylphthalate, dibutylsebacate, and the like.

Coating may be performed by applying the coating composition as a solution/suspension/blend using any conventional coating technique known in the prior art such as spray coating in a conventional coating pan or fluidized bed processor; dip coating or compression coating.

The coating composition may optionally include a portion of the dose of paliperidone.

Suitable solvents used as granulating fluid and for preparing solution/dispersion of coating substances include methylene chloride, isopropyl alcohol, acetone, methanol, ethanol, water and the like.

The following examples represent various embodiments according to the present invention. The examples are given solely for the purpose of illustration and are not to be construed as limitations of the present invention, as many variations thereof are possible without departing from the spirit and scope of the invention.

### EXAMPLE 1

| | | % w/w (wrt final tablet weight) | | | |
|---|---|---|---|---|---|
| S. No. | Ingredients | 1a | 1b | 1c | 1d |
| 1. | Paliperidone | 0.5-3 | 0.5-3 | 0.5-3 | 0.5-3 |
| 2. | Ethyl cellulose | 10-20 | 10-20 | | |
| 3. | Eudragit ® RS/RL/NM | | | 10-20 | 10-20 |
| 4. | Triethyl citrate | 0.5-3 | 0.5-3 | 0.5-3 | 0.5-3 |
| 5. | Polyvinyl pyrolidone | 2-5 | 2-5 | 2-5 | 2-5 |
| 6. | Isopropyl alcohol | q.s. to 6-10 | q.s. to 6-10 | q.s. to 6-10 | q.s. to 6-10 |
| 7. | Purified Water | q.s. to 6-10 | q.s. to 6-10 | q.s. to 6-10 | q.s. to 6-10 |
| 8. | Hydroxypropylmethyl cellulose | 30-60 | 30-60 | 30-60 | 30-60 |
| 9. | Lactose monohydrate | 20-40 | | 20-40 | |
| 10. | Microcrystalline cellulose | | 20-40 | | 20-40 |
| 11. | Polyvinyl pyrolidone | 3-8 | 3-8 | 3-8 | 3-8 |
| 12. | Magnesium stearate | 1-2 | 1-2 | 1-2 | 1-2 |
| 13. | Butylated hydroxyl toluene | 0.01-0.02 | 0.01-0.02 | 0.01-0.02 | 0.01-0.02 |

Extended Release Coating

| | | | | | |
|---|---|---|---|---|---|
| 14. | Ethyl cellulose | 5-10 | 5-10 | 5-10 | 5-10 |
| 15. | Triethyl citrate | 0.5-2 | 0.5-2 | 0.5-2 | 0.5-2 |
| 16. | Polyvinyl pyrolidone | 2-4 | 2-4 | 2-4 | 2-4 |
| 17. | Isopropyl alcohol | q.s. to 6-10 | q.s. to 6-10 | q.s. to 6-10 | q.s. to 6-10 |
| 18. | Purified water | q.s. to 6-10 | q.s. to 6-10 | q.s. to 6-10 | q.s. to 6-10 |

| | | | | |
|---|---|---|---|---|
| Final tablet weight | 100 | 100 | 100 | 100 |

### Process

1. Paliperidone was granulated using polyvinyl pyrrolidone in an isopropyl alcohol/water system in a GLATT fitted with a top spray assembly or alternatively in a Rapid Mixer Granulator.

2. The paliperidone granules of step 1 were granulated with an aqueous/non aqueous dispersion of ethyl cellulose (or) Eudragit® RS/RL/NM in a GLATT fitted with a top spray assembly or alternatively in a Rapid Mixer Granulator.
3. The granules of step 2 were then blended with butylated hydroxyl toluene, hydroxypropylmethyl cellulose, lactose monohydrate or microcrystalline cellulose and polyvinyl pyrrolidone in a low shear blender.
4. Step 3 blend was lubricated with magnesium stearate.
5. The powder blend of step 4 was compressed into tablets of suitable size.
6. The tablets of step 5 were coated with an aqueous/non aqueous or hydro-alcoholic dispersion of ethyl cellulose.

### EXAMPLE 2

| | | % w/w (wrt final tablet weight) | | | |
|---|---|---|---|---|---|
| S. No. | Ingredients | 2a | 2b | 2c | 2d |
| 1. | Paliperidone | 0.5-3 | 0.5-3 | 0.5-3 | 0.5-3 |
| 2. | Ethyl cellulose | 10-20 | 10-20 | | |
| 3. | Eudragit ® RS/RL/NM | | | 10-20 | 10-20 |
| 4. | Triethyl citrate | 0.5-3 | 0.5-3 | 0.5-3 | 0.5-3 |
| 5. | Polyvinyl pyrolidone | 2-5 | 2-5 | 2-5 | 2-5 |
| 6. | Isopropyl alcohol | q.s. to 6-10 | q.s. to 6-10 | q.s. to 6-10 | q.s. to 6-10 |
| 7. | Purified Water | q.s. to 6-10 | q.s. to 6-10 | q.s. to 6-10 | q.s. to 6-10 |
| 8. | Polyethylene oxide | 30-50 | 30-50 | 30-50 | 30-50 |
| 9. | Lactose monohydrate | 20-40 | | 20-40 | |
| 10. | Microcrystalline cellulose | | 20-40 | | 20-40 |
| 11. | Polyvinyl pyrolidone | 3-8 | 3-8 | 3-8 | 3-8 |
| 12. | Magnesium stearate | 1-2 | 1-2 | 1-2 | 1-2 |
| 13. | Butylated hydroxyl toluene | 0.01-0.02 | 0.01-0.02 | 0.01-0.02 | 0.01-0.02 |

Extended Release Coating

| | | | | | |
|---|---|---|---|---|---|
| 14. | Ethyl cellulose | 5-10 | 5-10 | 5-10 | 5-10 |
| 15. | Triethyl citrate | 0.5-2 | 0.5-2 | 0.5-2 | 0.5-2 |
| 16. | Polyvinyl pyrolidone | 2-4 | 2-4 | 2-4 | 2-4 |
| 17. | Isopropyl alcohol | q.s. to 6-10 | q.s. to 6-10 | q.s. to 6-10 | q.s. to 6-10 |
| 18. | Purified water | q.s. to 6-10 | q.s. to 6-10 | q.s. to 6-10 | q.s. to 6-10 |

Immediate Release Layer

| | | | | | |
|---|---|---|---|---|---|
| 19. | Paliperidone | 0.5-1 | 0.5-1 | 0.5-1 | 0.5-1 |
| 20. | Hydroxypropylmethyl cellulose | 3-5 | 3-5 | 3-5 | 3-5 |
| 21. | Sodium lauryl sulphate | 0.5-1 | 0.5-1 | 0.5-1 | 0.5-1 |
| 22. | Talc | 5-10 | 5-10 | 5-10 | 5-10 |

| | | | | |
|---|---|---|---|---|
| Final tablet weight | 100 | 100 | 100 | 100 |

### Process

1. Paliperidone was granulated using polyvinyl pyrrolidone in isopropyl alcohol/water system in a GLATT fitted with a top spray assembly or alternatively in a Rapid Mixer Granulator.
2. The paliperidone granules of step 1 were granulated with an aqueous/non aqueous dispersion of ethyl cellulose (or) Eudragit® RS/RL/NM in a GLATT fitted with a top spray assembly or alternatively in a Rapid Mixer Granulator.
3. The granules of step 2 were then blended with butylated hydroxyl toluene, polyethylene oxide, lactose monohydrate or microcrystalline cellulose and polyvinyl pyrrolidone in a low shear blender.
4. Step 3 blend was lubricated with magnesium stearate.
5. The powder blend of step 4 was compressed into tablets of suitable size.
6. The tablets of step 5 were coated with an aqueous/non aqueous or hydro-alcoholic dispersion of ethyl cellulose.
7. Ethyl cellulose coated tablets of step 6 were further coated with an immediate release Paliperidone layer of paliperidone, hydroxypropylmethyl cellulose, sodium lauryl sulphate and talc.

### EXAMPLE 3

| | | % w/w (wrt final tablet weight) | | | |
|---|---|---|---|---|---|
| S. No. | Ingredients | 3a | 3b | 3c | 3d |
| 1. | Paliperidone | 0.5-3 | 0.5-3 | 0.5-3 | 0.5-3 |
| 2. | Ethyl cellulose | 10-20 | 10-20 | | |
| 3. | Eudragit ® RS/RL/NM | | | 10-20 | 10-20 |
| 4. | Triethyl citrate | 0.5-3 | 0.5-3 | 0.5-3 | 0.5-3 |
| 5. | Polyvinyl pyrolidone | 2-5 | 2-5 | 2-5 | 2-5 |
| 6. | Isopropyl alcohol | q.s. to 6-10 | q.s. to 6-10 | q.s. to 6-10 | q.s. to 6-10 |
| 7. | Purified Water | q.s. to 6-10 | q.s. to 6-10 | q.s. to 6-10 | q.s. to 6-10 |
| 8. | Hydroxypropylmethyl cellulose | 30-50 | 30-50 | 30-50 | 30-50 |
| 9. | Lactose monohydrate | 20-40 | | 20-40 | |
| 10. | Microcrystalline cellulose | | 20-40 | | 20-40 |
| 11. | Polyvinyl pyrolidone | 3-8 | 3-8 | 3-8 | 3-8 |
| 12. | Magnesium stearate | 1-2 | 1-2 | 1-2 | 1-2 |
| 13. | Butylated hydroxyl toluene | 0.01-0.02 | 0.01-0.02 | 0.01-0.02 | 0.01-0.02 |

Extended Release Coating (compression coating)

| | | | | | |
|---|---|---|---|---|---|
| 14. | Ethyl cellulose | 10-20 | 10-20 | 10-20 | 10-20 |
| 15. | Polyethylene glycol | 5-10 | 5-10 | 5-10 | 5-10 |
| 16. | Butylated hydroxyl toluene | 0.01-0.02 | 0.01-0.02 | 0.01-0.02 | 0.01-0.02 |
| 17. | Magnesium stearate | 1-2 | 1-2 | 1-2 | 1-2 |
| 18. | Colloidal silica | 1-2 | 1-2 | 1-2 | 1-2 |

| | | | | |
|---|---|---|---|---|
| Final Tablet Weight | 100 | 100 | 100 | 100 |

### Process

1. Paliperidone was granulated using polyvinyl pyrrolidone in isopropyl alcohol/water system in a GLATT fitted with a top spray assembly or alternatively in a Rapid Mixer Granulator.
2. The paliperidone granules of step 1 were granulated with an aqueous/non aqueous dispersion of Ethyl cellulose (or) Eudragit® RS/RL/NM in a GLATT fitted with a top spray assembly or alternatively in a Rapid Mixer Granulator.
3. The granules of step 2 were then blended with butylated hydroxyl toluene, hydroxypropylmethyl cellulose, lactose monohydrate or microcrystalline cellulose and polyvinyl pyrrolidone in a low shear blender.
4. Step 3 blend was lubricated with magnesium stearate.
5. The powder blend of step 4 was compressed into tablets of suitable size.
6. The tablets of step 5 were compression coated with a composition of ethyl cellulose, polyethylene glycol, butylated hydroxyl toluene, magnesium stearate and colloidal silica.

### EXAMPLE 4

| | | % w/w (wrt final tablet weight) | | | |
|---|---|---|---|---|---|
| S. No. | Ingredients | 4a | 4b | 4c | 4d |
| 1. | Paliperidone | 0.5-3 | 0.5-3 | 0.5-3 | 0.5-3 |
| 2. | Ethyl cellulose | 10-20 | 10-20 | | |
| 3. | Eudragit ® RS/RL/NM | | | 10-20 | 10-20 |
| 4. | Triethyl citrate | 0.5-3 | 0.5-3 | 0.5-3 | 0.5-3 |
| 5. | Polyvinyl pyrolidone | 2-5 | 2-5 | 2-5 | 2-5 |
| 6. | Isopropyl alcohol | q.s. to 6-10 | q.s. to 6-10 | q.s. to 6- | q.s. to 6- |
| 7. | Purified Water | q.s. to 6-10 | q.s. to 6-10 | q.s. to 6-10 | q.s. to 6-10 |
| 8. | Hydroxypropylmethyl cellulose | 30-50 | 30-50 | 30-50 | 30-50 |
| 9. | Lactose monohydrate | 20-40 | | 20-40 | |
| 10. | Microcrystalline cellulose | | 20-40 | | 20-40 |
| 11. | Polyvinyl pyrolidone | 3-8 | 3-8 | 3-8 | 3-8 |
| 12. | Magnesium stearate | 1-2 | 1-2 | 1-2 | 1-2 |
| 13. | Butylated hydroxyl toluene | 0.01-0.02 | 0.01-0.02 | 0.01-0.02 | 0.01-0.02 |

Extended Release Coating (compression coating)

| | | | | | |
|---|---|---|---|---|---|
| 14. | Polyethylene oxide | 10-20 | 10-20 | 10-20 | 10-20 |
| 15. | Polyethylene glycol | 5-10 | 5-10 | 5-10 | 5-10 |
| 16. | Butylated hydroxyl toluene | 0.01-0.02 | 0.01-0.02 | 0.01-0.02 | 0.01-0.02 |
| 17. | Magnesium stearate | 1-2 | 1-2 | 1-2 | 1-2 |
| 18. | Colloidal silica | 1-2 | 1-2 | 1-2 | 1-2 |

| | | | | |
|---|---|---|---|---|
| Final Tablet Weight | 100 | 100 | 100 | 100 |

### Process

1. Paliperidone was granulated using polyvinyl pyrrolidone in isopropyl alcohol/water system in a GLATT fitted with a top spray assembly or alternatively in a Rapid Mixer Granulator.
2. The paliperidone granules of step 1 were granulated with an aqueous/non aqueous dispersion of ethyl cellulose (or) Eudragit® RS/RL/NM in a GLATT fitted with a top spray assembly or alternatively in a Rapid Mixer Granulator.
3. The granules of step 2 were then blended with butylated hydroxyl toluene, hydroxypropylmethyl cellulose, lactose monohydrate or microcrystalline cellulose and polyvinyl pyrrolidone in a low shear blender.
4. Step 3 blend was lubricated with magnesium stearate.
5. The powder blend of step 4 was compressed into tablets of suitable size.
6. The tablets of step 5 were compression coated with a composition of polyethylene oxide, polyethylene glycol, butylated hydroxyl toluene, magnesium stearate and colloidal silica.

1. An extended release pharmaceutical composition comprising paliperidone and one or more release controlling polymers in a matrix formulation, coated with one or more delayed release coatings.

2. The extended release pharmaceutical composition of claim 1, wherein the paliperidone is present in an amount of about 0.5% to about 10% w/w of the total composition.

3. The extended release pharmaceutical composition of claim 1, wherein the paliperidone particles have a D₅₀ of about 1 μm to about 10 μm and a D₉₀ of about 2 μm to about 30 μm.

4. The extended release pharmaceutical composition of claim 1, wherein the one or more release controlling polymer comprises a water insoluble polymer.

5. The extended release pharmaceutical composition of claim 1, wherein the one or more release controlling polymer are present in an amount of about 10% to about 90% w/w of the total composition.

6. The extended release pharmaceutical composition of claim 5, wherein the release controlling polymer comprises one or more of ethyl cellulose, hydroxyethylcellulose, cellulose acetate, cellulose acetate butyrate, cellulose acetate phthalate, cellulose acetate trimellitate, hydroxypropyl methylcellulose phthalate, poly (alkyl) methacrylate, and copolymers of acrylic or methacrylic acid esters, ammonio methyacrylate copolymer, methyacrylic acid copolymers, methacrylic acid-acrylic acid ethyl ester copolymer, methacrylic acid esters neutral copolymer, dimethylaminoethylmethacrylate-methacrylic acid esters copolymer, vinyl methyl ether/maleic anhydride copolymers, their salts and esters, polyvinyl acetate and mixtures thereof.

7. The extended release pharmaceutical composition of claim 1, further comprising one or more pharmaceutically inert excipients selected from the group comprising of solubility enhancers/solubilizers, fillers, binders, lubricants/glidants, coloring agents, plasticizers and opacifiers.

8. The extended release pharmaceutical composition of the claim 1, further comprising one or more non-functional coating layers.

9. A process for the preparation of the extended release pharmaceutical composition of claim 1, wherein said process comprises the steps of:
(a) blending the paliperidone, the one or more release controlling polymers with one or more pharmaceutically inert excipients;
(b) optionally granulating the blend of step (a);
(c) compressing the blend/granules of steps (a) or (b) into tablets using appropriate tooling; and
(d) applying one or more delayed release coatings on to the compressed tablets of step (c).

10. A process for the preparation of the extended release pharmaceutical composition of claim 1, wherein said process comprises the steps of:
(a) granulating the paliperidone with the one or more release controlling polymers, with or without one or more pharmaceutically inert excipients;
(b) mixing the granules with the one or more release controlling polymers and one or more pharmaceutically inert excipients;
(c) compressing the granules into tablets using appropriate tooling; and
(d) applying the one or more delayed release coatings on to the compressed tablets of step (c).

11. A process for the preparation of the extended release pharmaceutical composition of claim 1, wherein said process comprises the steps of:
(a) granulating the paliperidone with the one or more release controlling polymers, with or without one or more pharmaceutically inert excipients;
(b) mixing the granules with the one or more release controlling polymers and one or more pharmaceutically inert excipients;
(c) compressing the granules into tablets using appropriate tooling;
(d) applying the one or more delayed release coatings on to the compressed tablets of step (c);
(e) mixing the paliperidone with one or more coating additives; and
(f) applying the mixture of step (e) on to the coated tablets of step (d).

12. A process for the preparation of the extended release pharmaceutical composition of claim 1, wherein said process comprises the steps of:
(a) blending the paliperidone and the one or more release controlling polymer with one or more pharmaceutically inert excipients;
(b) optionally granulating the blend of step (a);
(c) compressing the blend/granules of steps (a) or (b) into tablets using appropriate tooling; and
(d) applying the compression coating of the one or more delayed release coatings on to the compressed tablets of step (c).

13. A process for the preparation of the extended release pharmaceutical composition of claim 1, wherein said process comprises the steps of:
(a) blending the paliperidone with one or more pharmaceutically inert excipients;
(b) optionally granulating the blend of step (a);
(c) compressing the blend/granules of steps (a) or (b) into tablets using appropriate tooling;
(d) mixing the paliperidone with the one or more release controlling polymers and one or more coating additives; and
(e) applying the compression coating of the mixture of step (d) on to the compressed tablets of step (c).

14. A process for the preparation of the extended release pharmaceutical composition of claim 1, wherein said process comprises the steps of:
(a) blending the paliperidone with one or more pharmaceutically inert excipients;
(b) optionally granulating the blend of step (a);
(c) compressing the blend/granules of steps (a) or (b) into tablets using appropriate tooling; and
(d) applying a compression coating of the one or more delayed release coatings on to the compressed tablets of step (c).

15. The extended release pharmaceutical composition of claim 1, wherein said composition releases the paliperidone at a rate of :
(a) not more than 20% after 2 hours;
(b) between 10 and 40% after 8 hours;
(c) between 40 and 80% after 14 hours;
(d) between 70 and 95% after 18 hours; and
(e) more than 80% after 24 hours.

16. A method of treatment of neurological disorders in mammals comprising administering to a mammal in need thereof, an extended release composition comprising paliperidone or pharmaceutically acceptable salts, enantiomers, hydrates, solvates, metabolites, prodrugs or mixture thereof in one or more release controlling polymers, coated with one or more delayed release coatings.
